(19) United States
(12) Patent Application Publication
(10) Pub. No.: US 2011/0201882 A1
(43) Pub. Date: Aug. 18, 2011

(54) Endotracheal tube exchanger and detachable stylet assembly therefor

(75) Inventors: John Schwartz, Williamston, MI (US); Richard Schwartz, Evans, GA (US); Harsha Setty, Martinez, GA (US)

(73) Assignee: AI Medical Devices, Inc., Williamston, MI (US)

(21) Appl. No.: 12/658,959

(22) Filed: Feb. 18, 2010

(51) Int. Cl. A61B 1/267; A61B 1/04; A61M 16/04

(52) U.S. Cl. 600/109; 600/120

(57) ABSTRACT

An endotracheal tube exchanger with a detachable stylet, comprising a support housing and a detachable stylet assembly removably interconnected with the support housing. The stylet assembly includes an elongated stylet tube having a distal end and a proximal end, the distal end having provided therein a camera and at least one LED, and the proximal end terminating in an end cap electrically connected with the camera and at least one LED. The end cap receivable in the support housing and adapted for electrical interconnection therewith.

### CROSS-REFERENCE TO RELATED APPLICATION

Not applicable.

### STATEMENT REGARDING FEDERALLY SPONSORED RESEARCH OR DEVELOPMENT

Not applicable.

### FIELD OF THE INVENTION

The present invention pertains to endotracheal tube exchangers, and more particularly to an endotracheal tube exchnger with a detachable stylet assembly.

### BACKGROUND

Currently, endotracheal tube exchangers are utilized in operating room and intensive-care unit settings in order to replace previously positioned endotracheal tubes. Reasons for changing such endotracheal tubes include, by way of non-limiting example, damage to the endotracheal cuff, incorrect dimensions for the endotracheal tube, airway fire, and extended intubation with the same tube. Conventionally, endotracheal tube exchange is accomplished by placing a central lumen or catheter through the previously-positioned endotracheal tube, and inserting that central lumen or catheter a sufficient distance into the endotracheal tube so that it is presumably positioned within the patient's trachea. The existing endotracheal tube is then deflated and removed from the trachea over the catheter, which is used as a guide. A new endotracheal tube is then inserted into the trachea over the catheter and advanced into position. The catheter or central lumen is then withdrawn and proper placement of the endotracheal tube is subsequently confirmed by the use of a fiber-optic bronchoscope.

### SUMMARY OF THE DISCLOSURE

There is disclosed an endotracheal tube exchanger with a detachable stylet assembly.

According to a first embodiment, the detachable stylet assembly comprises an elongated stylet tube having a distal end and a proximal end, the distal end having provided therein a camera and at least one LED, and the proximal end terminating in an end cap electrically connected with the camera and at least one LED. The end cap is adapted for removable electrical interconnection with a support housing.

Per one feature thereof, the stylet tube includes a curved section proximate the distal end thereof.

According to another feature, the end cap comprises a plurality of discrete contacts.

Per still another feature, the stylet assembly further comprises a PC board disposed in the stylet tube, the PC board positioned between, and in electrical contact with, each of the plurality of discrete contacts in the end cap and the camera and at least one LED proximate the distal end.

Per one feature, the end cap is removably mechanically connectable to the support housing.

According to another feature, the end cap is notched for physical interconnection with a support housing.

The support housing includes, according to one embodiment thereof, an electrical connection port in which is matingly receivable the end cap of the stylet tube.

Per one feature of the invention, the electrical connection port comprises a plurality of electrical contacts for electrical interconnection with the end cap.

According to another aspect of the invention, a display monitor is mounted relative to the support housing, the display monitor being electrically connected through the support housing to the plurality of electrical contacts of the electrical connection port.

Per yet another feature, the display monitor houses a power source for powering the display monitor and the camera and at least one LED disposed in the distal end of the stylet tube.

According to a still further feature, the display monitor is removably mounted on an electrical connector provided on the support housing, the electrical connector being electrically connected through the support housing to the plurality of electrical contacts of the electrical connection port

Per another feature of the invention, the endotracheal tube exchanger may further comprise a hand grip connected to the support housing.

According to a still further feature, the support housing is removably connected to the hand grip.

An exemplary method employing the present invention to remove a used endotracheal tube from a patient and insert a new endotracheal tube in its place comprises the steps of:

(a) providing an endotracheal tube exchanger including a support housing having a display monitor mounted relative thereto; and a detachable stylet assembly removably interconnected with the support housing, the stylet assembly including an elongated stylet tube having a distal end and a proximal end, the distal end having provided therein a camera and at least one LED, and the proximal end terminating in an end cap electrically connected with the camera and at least one LED, the end cap receivable in the support housing and adapted for electrical interconnection therewith, whereby the display monitor is electrically connected with the camera and at least one LED provided in the distal end of the stylet tube;

(b) using the endotracheal tube exchanger to insert the stylet tube into a first endotracheal tube disposed in the trachea of a patient;

(c) disconnecting the stylet tube from the hand grip;

(d) removing the first endotracheal tube from the patient over the stylet tube;

(e) replacing the first endotracheal tube with a second endotracheal tube over the stylet tube;

(f) reconnecting the stylet tube to the hand grip;

(g) visualizing and adjusting as necessary the position of the second endotracheal tube using the display monitor on the hand grip and the at least one LED and camera provided adjacent the distal end of the stylet tube; and

(h) removing the stylet tube from the second endotracheal tube.

### BRIEF DESCRIPTION OF THE DRAWINGS

The present invention will be appreciated from the following description and accompanying drawings, of which:

FIG. 1 is a discontinuous lateral view of a stylet tube according to the present invention.

FIG. 2 is a discontinuous lateral, cross-sectional view of a stylet tube according to the present invention.

FIG. 3 is a distal-end view of the stylet tube of FIG. 2, taken along lines 3-3 thereof.

FIG. 4 is a schematic depiction of the electrical connection among the various elements of the present invention, including the camera, PC board, end cap, support housing contacts and electrical connector.

FIG. 5 is a top-down view of the stylet support housing of the present invention.

FIG. 6 is a lateral, cut-away view of the stylet support housing of FIG. 5, taken along lines 6-6 thereof.

FIG. 7 is a top-down view of the stylet support housing of the present invention, further showing a stylet connected thereto.

FIG. 8 is lateral, cut-away view of the stylet support housing of FIG. 7, taken along lines 8-8.

FIGS. 9 and 10 depict the support housing with a stylet tube and display monitor shown connected thereto.

FIGS. 11 through 15 sequentially depict the method of employing the present invention in replacing a first, used endotracheal tube with a second, new endotracheal tube.

### WRITTEN DESCRIPTION

All patents, patent applications, government publications, government regulations, and literature references cited in this specification are hereby incorporated herein by reference in their entirety, including, without limitation, the disclosures of the following US patents and applications: U.S. Pat. No. 7,658,708, issued 9 Feb. 2009; U.S. Pat. No. 7,458,375, issued 2 December 2008; Ser. No. 11/820,117, filed 18 Jun. 2007; Ser. No. 11/906,870, filed 4 Oct. 2007; Ser. No. 12/148,033, filed 16 Apr. 2008; Ser. No. 12/148,050, filed 16 Apr. 2008; and Ser. No. 12/587,905, filed 15 Oct. 2009. In case of conflict, the present description, including definitions, will control.

As required, detailed embodiments of the present invention are disclosed herein. However, it is to be understood that the disclosed embodiments are merely exemplary of the invention that may be embodied in various and alternative forms. The accompanying drawings are not necessarily to scale, and some features may be exaggerated or minimized to show details of particular components. Therefore, specific structural and functional details disclosed herein are not to be interpreted as limiting, but merely as a representative basis for teaching one skilled in the art to variously employ the present invention.

Referring now to the drawings, and more particularly to FIGS. 1-3, there is disclosed in a first embodiment of the invention a detachable stylet assembly for an endotracheal tube exchanger (not shown in FIGS. 1-3), the assembly comprising an elongated stylet 100 having a distal end 101 and a proximal end 102.

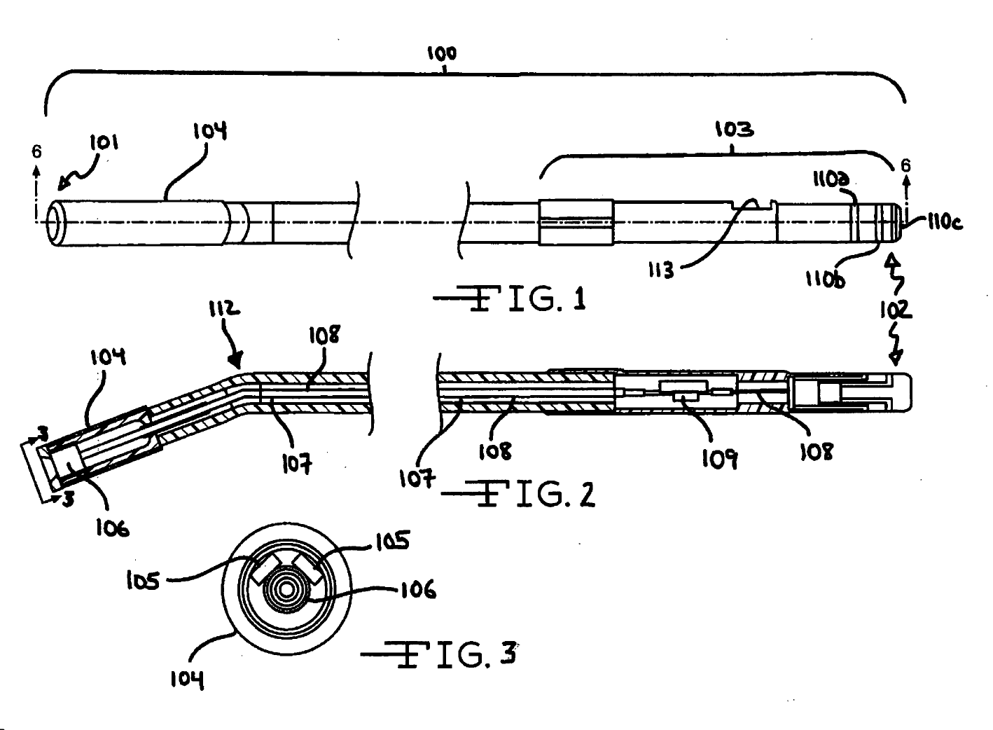

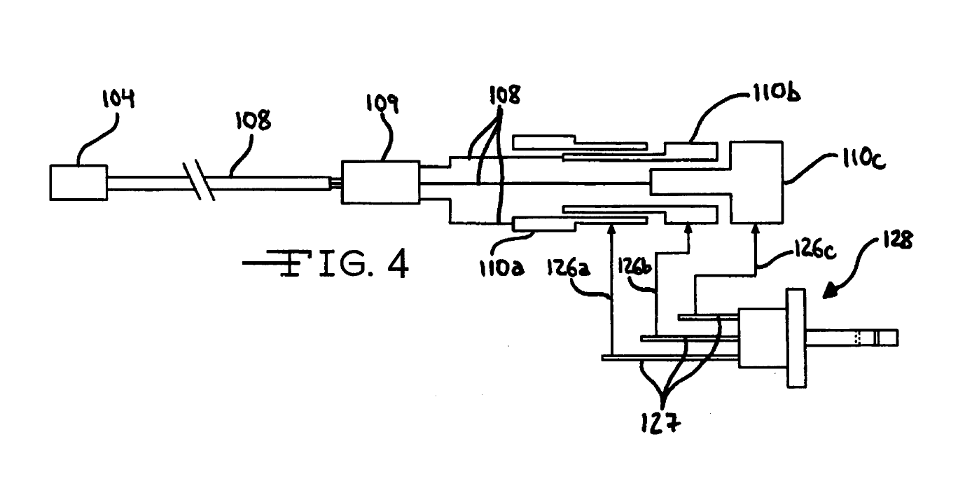

The proximal end 102 terminates in an end cap 103 adapted for removable, electrical interconnection with a support housing 120 (not shown in FIGS. 1-3). Distal end terminates in a housing 104 for at least one LED light 105 (two such LEDs are shown in the exemplary embodiment) and a camera 106 (shown in FIGS. 2 and 3), such as, in the exemplary embodiment, a CMOS chip having optics. Each such LED light 105 is positioned to illuminate an area proximate the distal end 101 of stylet 100, while camera 106 is adapted to provide the device's operator with a visual image of such area.

As best shown in FIG. 2, stylet 100 is an elongate tube defining an internal passageway 107. Camera 106 and the at least one LED light 105 are electrically connected to end cap 103 defined at proximal end 102 via wires 108 extending through the interior passageway 107 of stylet tube 100 and interconnected with in-line PC board 109 (e.g., a conventional voltage regulator board), which, in turn, is electrically connected to contacts 110a-110c defined on end cap 103.

Referring now to FIG. 2 in particular, distal end 101 will be seen to include a curved section 112 proximate thereto, which curved section 112 is of a radius adapted to facilitate the insertion and removal of endotracheal tubes relative to the stylet 100.

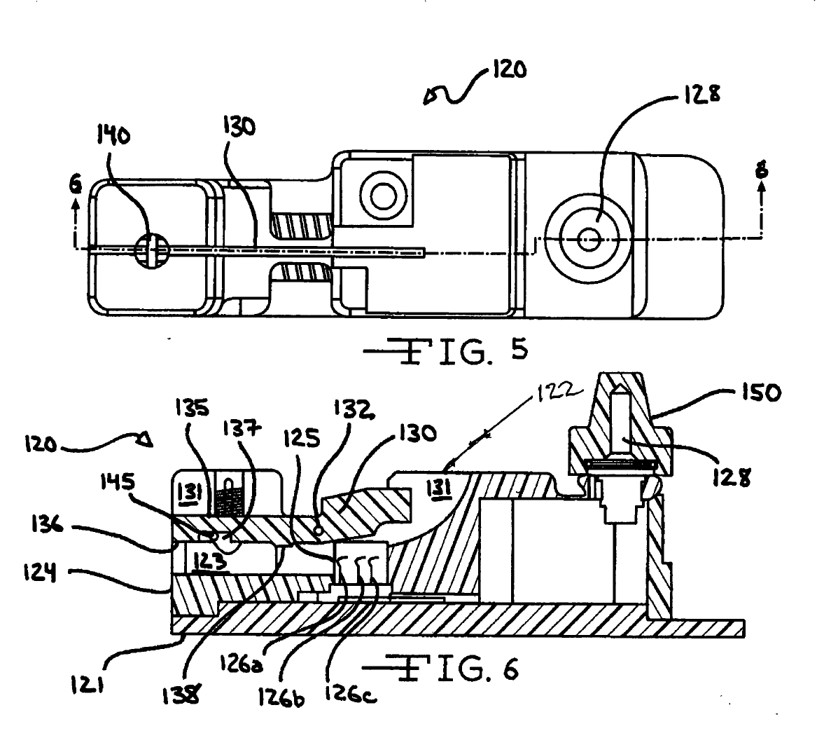

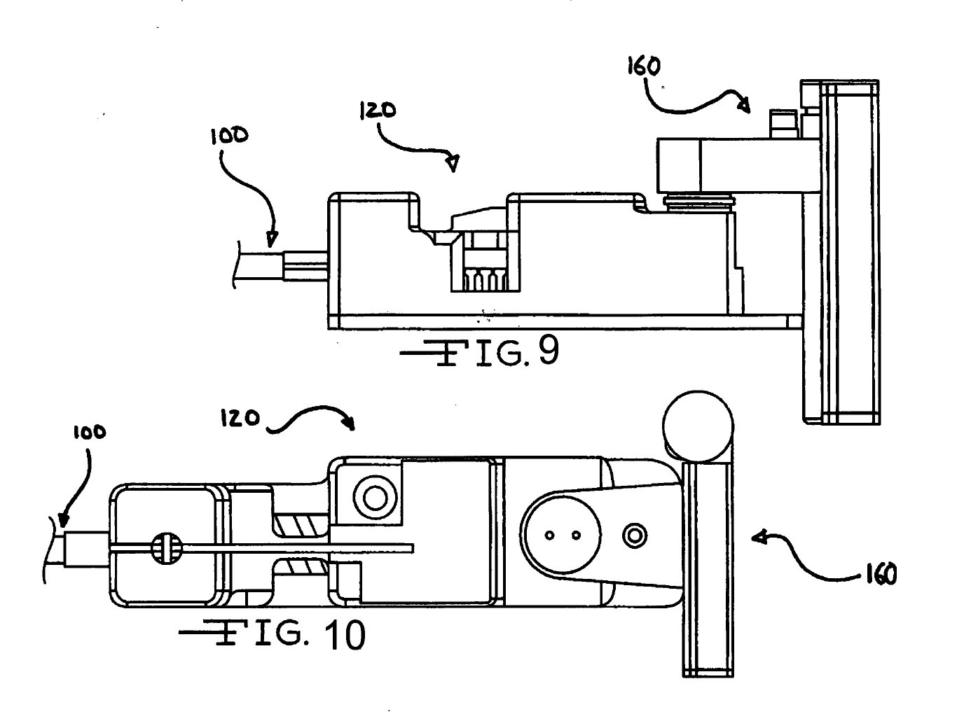

Referring then to FIGS. 5-8, stylet 100 is, as noted, adapted for removable interconnection with a support housing 120 to define, in combination, an endotracheal tube exchanger. Support housing 120 defines overall a substantially rectangular shape, according to the exemplary embodiment, although such overall shape is not intended to be limiting of the invention.

It is contemplated that support housing 120 may be dimensioned, such as in the illustrated embodiment, so as to be capable of being hand-held without further modification thereto. Alternatively, it is contemplated that support housing 120 may be provided with one or more gripping portions (not shown in FIGS. 5-8) disposed thereon, these gripping portions adapted to make support housing 120 more comfortably hand-held by a user.

With continuing reference to FIGS. 5-8, support housing 120 according to the illustrated embodiment includes a back plate 121 secured to a housing block 122. An electrical connection port 123 dimensioned to receive therein at least a terminal portion of the end cap 103 of stylet 100 is defined in housing block 122 opens onto an end face 124 of the support housing 120 and terminates at an end wall 125. Proximate to end wall 125 are provided a plurality of electrical contacts 126a-126c. Electrical contacts 126a-126c are in electrical connection (such as via electrical wiring 127 shown in FIG. 8) with an electrical connector 128 mounted (and, preferably though not necessarily, potted against rotation) in the housing block 122 and projecting outwardly and away from the support housing 120. As shown, electrical contacts 126a-126c comprise spring-type contacts for contacting respective contacts 110a-110c on end cap 103. However, it will be appreciated by those skilled in the art that other types of contacts may be employed for completing electrical connection with the end cap 103 of stylet 100.

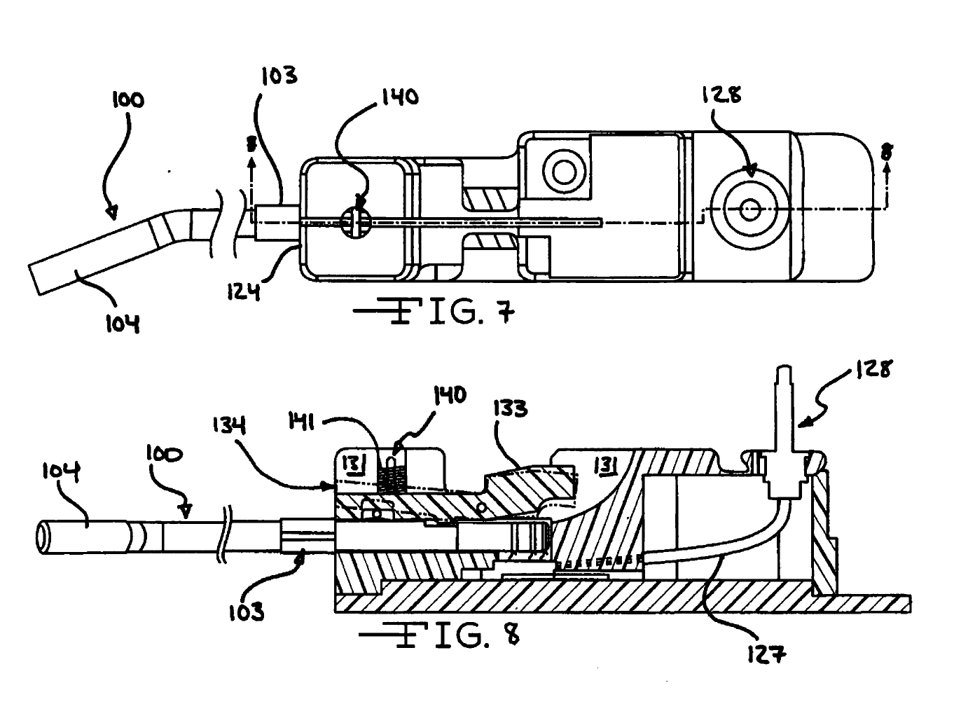

Optionally, a protective cap 150 (shown in FIG. 6) may be provided, protective cap 150 being removably receivable over with electrical connector 128 to protect the electrical connector when not in use, during sterilization of the support housing 120, etc.

Referring next to FIG. 4, there is depicted schematically the electrical connection between electrical connector 128, camera and the at least one LED in housing 104 via the contacts 126a-126c, contacts 110a-110c in end cap 103, wiring 108, wiring 127 and PC board 109. In particular, it may be seen from FIG. 4 that contacts 111f-111g, which correspond, respectively, to the video connection and a 5V DC power connection, are connected with contacts 110b and 111c via wires 108, while contact 111h, which corresponds to a ground connection, is connected with contact 111a via a further wire 108. In turn, contacts 126b and 126c, each of which is associated with connector 128 via wires 127, interconnect with, respectively, contacts 110b and 110c when end cap 103 is fully seated in electrical connection port 123; while contact 126a, which is likewise associated with connector 128 via wire 127, interconnects with contact 110a.

With reference again being had to FIGS. 5-8, manual release lever 130 is mounted on housing block 122 proximate electrical connection port 123 to facilitate the selective connection and disconnection of stylet 100 relative to the support housing 120. More particularly, release lever 130 comprises a thin, elongate element disposed within a slot 131 defined in the housing block 122 and in communication with the electrical connection port 123. Release lever 130 is pivotably mounted on a pin 132 disposed perpendicularly to the longitudinal axis of electrical connection port 123. A first end of release lever 130 defines a region 133 for a user to push release lever 130 downwardly, forcing the opposite end 134 upwardly (shown in phantom lines in FIG. 8). As shown in FIGS. 6 and 8, slot 131 is configured to provide sufficient clearance for the downward movement of release lever 130 proximate region 133.

With continuing reference to FIGS. 6 and 8, a spring-loaded stop 140 disposed within housing block 122 above an upper edge 135 of release lever 130 is positioned to contact the upper edge 135 as the opposite end 134 moves upwardly during pivotable movement of the lever. The spring 141 is biased to urge the stop 140, and thus the lever 130, back to the default state or condition (shown in FIG. 6) once a user ceases pushing on the level at region 133.

Along a lower edge 136, release lever 130 includes a cut-out or recess 137 which contacts a stop 145 that prevents lever 130 from being urged downwardly beyond the default state (FIG. 6) thereof. Between recess 137 and pivot pin 132 a lock tab 138 is provided on lower edge 136, the lock tab projecting away from the lower edge and toward the electrical connection port 123.

Referring again to FIG. 1, the means for physically interconnecting the stylet 100 and support housing 120 of the hand grip may be more fully understood. More particularly, a cut-out or slot 113 is defied in cap 103 of stylet 100 adjacent proximal end 102. Upon insertion of cap 103 into electrical connection port 123, release lever 130 is temporarily pivoted upwardly by the engagement of lock tab 138 with the end cap 103. When, with continued insertion of end cap 103 into the port 123, lock tab 138 is positioned adjacent slot 113, lock tab 138 is urged into engagement with slot 113 by the biasing action of spring-loaded stop 140. In this condition, stylet 100 is physically connected to support housing 120, and contacts 110a-110c of end cap 103 are in full electrical connection with contacts 126a-126c. Specifically, the stylet 100 is physically interlocked with the housing 120, and the two units remain physically and electrically connected until disengaged by a user (e.g., via actuation of the release lever 130).

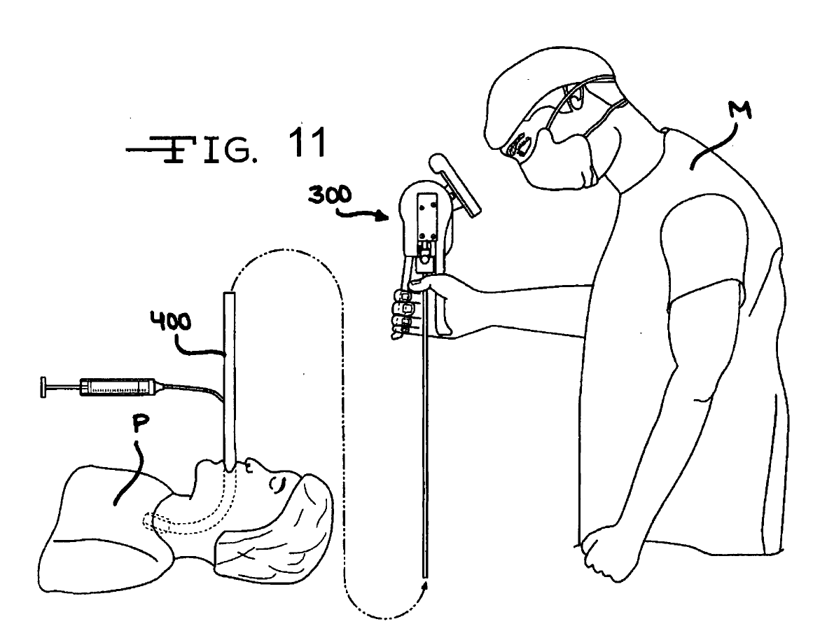

Turning next to FIGS. 9 and 10, a display monitor 160 is mountable on electrical connector 128. Monitor 160 can be pivotably mountable on electrical connector 128 to allow for rotation to a desired viewing position. Monitor 160 can be any viewing monitor, such as, by way of non-limiting example, an LCD screen. In FIG. 9, the mounted monitor 160 is shown from the bottom up, while in FIG. 10 the monitor 160 is shown from the side.

Further according to the illustrated embodiment, display monitor 160 also houses a 5V power source (not shown) for powering the camera 106 and the at least one LED 105 disposed in housing 104 of stylet 100, as well as the display monitor 160 itself. Of course, it is further contemplated that such power could be supplied by an external source of conventional design, including, by way of non-limiting example, an external battery, power cord to an electrical outlet, etc.

As heretofore described, electrical connection is provided between electrical connector 128 and electrical contacts 126a-126c via electrical wiring 127, by which it will be appreciated that, on receipt of at least a terminal portion of end cap 103 of stylet 100 in electrical connection port 123, electrical connection will be complete between, on one end, the camera 106 and at least one LED 105 provided in housing 104 at distal end 101 of stylet 100 and, on the other end, display monitor 160 and power source.

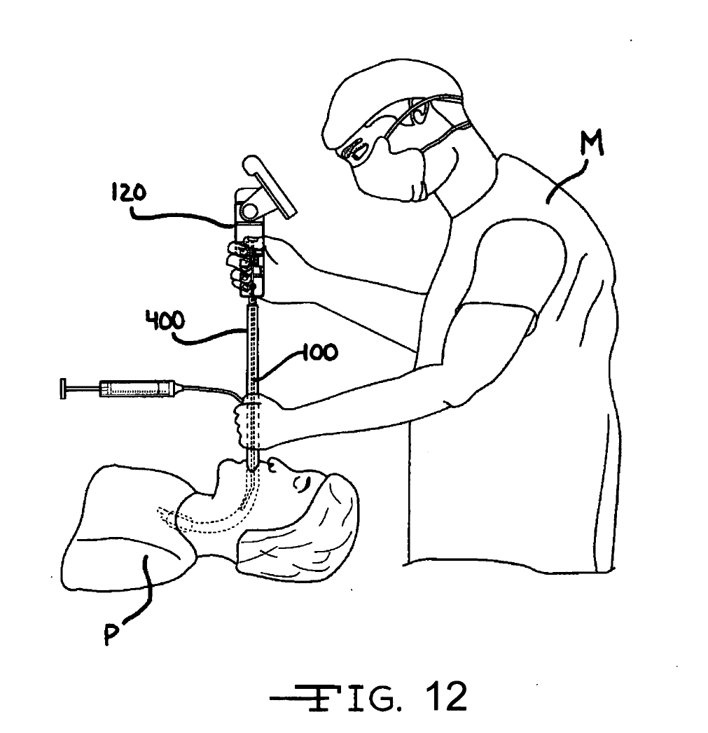

In use, the endotracheal tube exchanger as heretofor described may be employed, by way of non-limiting example, to remove a used endotracheal tube from a patient and insert a new endotracheal tube in its place. According to such method, the endotracheal tube to be replaced may have been inserted into the patient according to any conventional means including, by way of example and without limitation, through the use of any of the endoscopes disclosed in the patent applications referenced above and incorporated herein. So much is depicted in FIG. 11, which shows a medical practitioner M removing an endoscopic device 300 from a patient P after having used the device to place a first endotracheal tube 400 in the patient P.

When, for any reason, it becomes necessary to replace the first endotracheal tube 400, the tube exhanger of the present invention is employed to insert the stylet tube 100 into the first endotracheal tube 400. This step is shown in FIG. 12. With the stylet 100 positioned as desired within the first endotracheal tube 400, the stylet 100 is disconnected from the support housing 120. More particularly, the user pushes lever 130 to release tab 138 from slot 113, all in the manner heretofore described, thereby permitting stylet tube 100 to be disengaged and withdrawn from the electrical connection port 123.

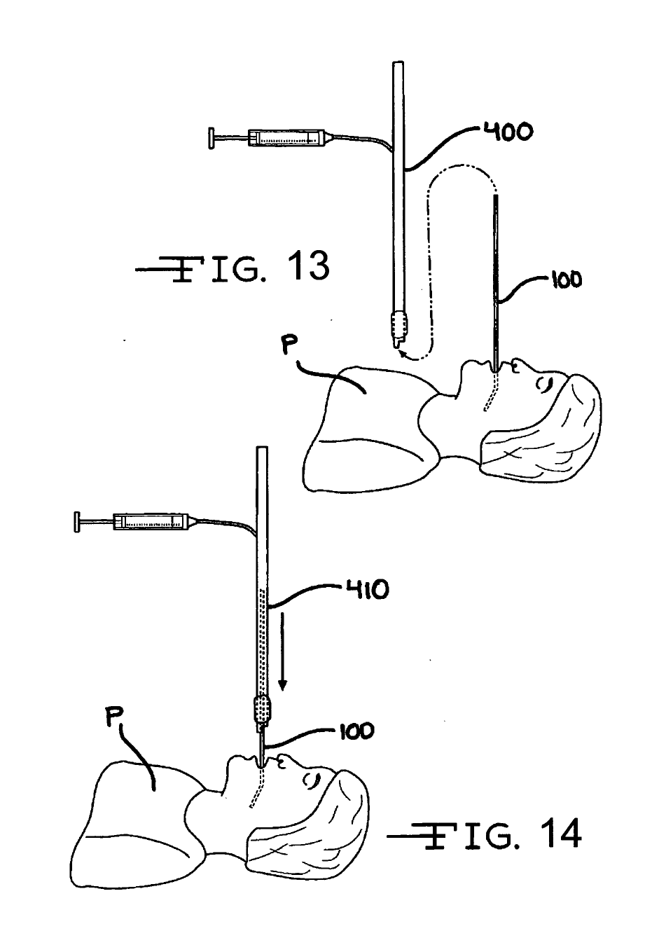

With the stylet tube 100 thus disconnected from the support housing 120, the first endotracheal tube 400 may be withdrawn from the patient P over the stylet tube 100 free of any obstruction by the support housing 120. This step is shown in FIG. 13.

Following withdrawal of the first endotracheal tube 400 over the stylet tube 100, a second, replacement endotracheal tube 410 may be more easily inserted into the patient P using the previously positioned stylet tube 100 as a guide. More particularly, the medical practitioner M inserts the second endotracheal tube 410 over the stylet tube 100, thereafter advancing the endotracheal tube along the stylet 100 to insert the endotracheal tube 410 into the patient P. This step is shown in FIG. 14.

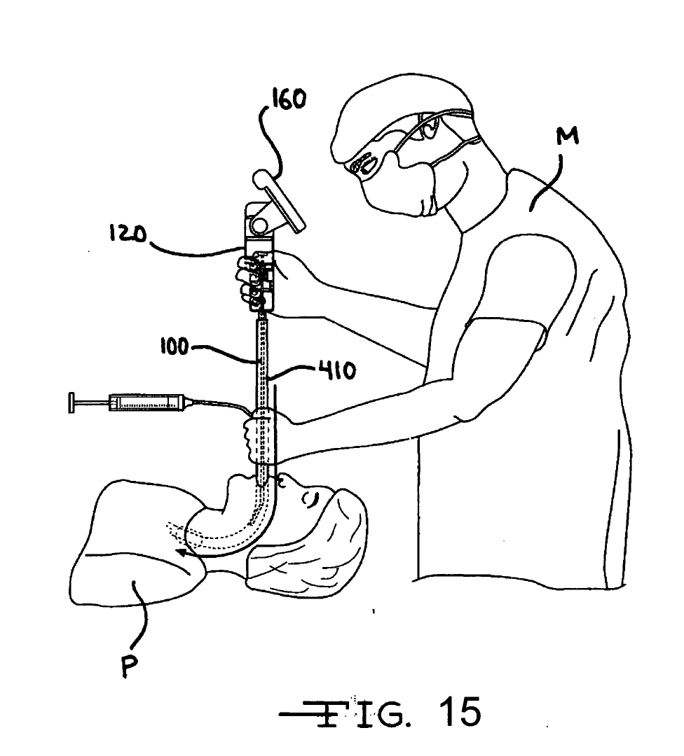

Once the second endotracheal tube 410 has been inserted, the stylet tube 100 is reconnected to the support housing 120 in the manner heretofore described. With the stylet tube 100 thus reconnected, it will be appreciated that the camera provided at the distal end of the stylet tube 100 will once again be in electrical connection with the display monitor 160, such that the medical practitioner M may thereafter employ the tube exchanger to visualize and, as necessary, adjust the position of the second endotracheal tube 410 using the images on the display monitor 160 provided by the camera 106. This step is shown in FIG. 15.

In the final step of this method (not shown), the stylet tube 100 is removed from the patient P once the second endotracheal tube 410 is in a desired position within the patient P. Specifically, the medical practitioner M can hold the replacement endotracheal tube 410 with one hand and remove the interconnected stylet 100 and housing 120 by gripping the housing 120 with the other hand.

By the foregoing, it will be appreciated that each endotracheal tube placed in a patient can be readily removed and replaced with another endotracheal tube using but a single device. Accordingly, the present invention does away with the need for the provision of separate apparatus—i.e., the catheter and fiber-optic bronchoscope—to facilitate both the initial placement and finer positioning of replacement endotracheal tubes.

The foregoing description of the exemplary embodiment of the invention has been presented for purposes of illustration and description. It is not intended to be exhaustive or to limit the invention to the precise form disclosed, and modifications and variations are possible in light of the above teachings or may be acquired from practice of the innovation. The embodiments are shown and described in order to explain the principals of the innovation and its practical application to enable one skilled in the art to utilize the innovation in various embodiments and with various modifications as are suited to the particular use contemplated. Although only a few embodiments of the present innovations have been described in detail in this disclosure, those skilled in the art who review this disclosure will readily appreciate that many modifications are possible without materially departing from the novel teachings and advantages of the subject matter recited. Accordingly, all such modifications are intended to be included within the scope of the present innovations. Other substitutions, modifications, changes and omissions may be made in the design, operating conditions and arrangement of the exemplary embodiments without departing from the spirit of the present innovations.

We claim:

1. A detachable stylet assembly for an endotracheal tube exchanger, comprising an elongated stylet tube having a distal end and a proximal end, the distal end having provided therein a camera and at least one LED, and the proximal end terminating in an end cap electrically connected with the camera and at least one LED, the end cap adapted for removable electrical interconnection with a support housing.

2. The detachable stylet assembly of claim 1, wherein the stylet tube includes a curved section proximate the distal end thereof.

3. The detachable stylet assembly of claim 1, wherein the end cap comprises a plurality of discrete contacts.

4. The detachable stylet assembly of claim 3, further comprising a PC board disposed in the stylet tube, the PC board positioned between, and in electrical contact with, each of the plurality of discrete contacts in the end cap and the camera and at least one LED proximate the distal end.

5. The detachable stylet assembly of claim 1, wherein the end cap is notched for physical interconnection with a support housing.

6. An endotracheal tube exchanger, comprising:
a support housing; and
a detachable stylet assembly removably interconnected with the support housing, the stylet assembly including an elongated stylet tube having a distal end and a proximal end, the distal end having provided therein a camera and at least one LED, and the proximal end terminating in an end cap electrically connected with the camera and at least one LED, the end cap receivable in the support housing and adapted for electrical interconnection therewith.

7. The endotracheal tube exchanger of claim 6, wherein the stylet tube includes a curved section proximate the distal end thereof.

8. The endotracheal tube exchanger of claim 6, wherein the end cap comprises a plurality of discrete contacts.

9. The endotracheal tube exchanger of claim 8, further comprising a PC board disposed in the stylet tube, the PC board positioned between, and in electrical contact with, each of the plurality of discrete contacts in the end cap and the camera and at least one LED proximate the distal end.

10. The endotracheal tube exchanger of claim 6, wherein the end cap is matingly receivable in an electrical connection port defined in the support housing.

11. The endotracheal tube exchanger of claim 10, wherein the electrical connection port comprises a plurality of electrical contacts for electrical interconnection with the end cap.

12. The endotracheal tube exchanger of claim 10, wherein the end cap is removably mechanically connectable to the support housing.

13. The endotracheal tube exchanger of claim 6, further comprising a display monitor mounted relative to the support housing, wherein the display monitor is electrically connected through the support housing to the plurality of electrical contacts of the electrical connection port.

14. The endotracheal tube exchanger of claim 13, wherein the display monitor houses a power source for powering the display monitor and the camera and at least one LED disposed in the distal end of the stylet tube.

15. The endotracheal tube exchanger of claim 13, wherein the display monitor is removably mounted on an electrical connector provided on the support housing, the electrical connector being electrically connected through the support housing to the plurality of electrical contacts of the electrical connection port

16. The endotracheal tube exchanger of claim 6, further comprising a hand grip connected to the support housing.

17. The endotracheal tube exchanger of claim 16, wherein the support housing is removably connected to the hand grip.

18. A method for removing a used endotracheal tube from a patient and inserting a new endotracheal tube in its place, comprising the steps of:
(a) providing an endotracheal tube exchanger including a support housing having a display monitor mounted relative thereto; and a detachable stylet assembly removably interconnected with the support housing, the stylet assembly including an elongated stylet tube having a distal end and a proximal end, the distal end having provided therein a camera and at least one LED, and the proximal end terminating in an end cap electrically connected with the camera and at least one LED, the end cap receivable in the support housing and adapted for electrical interconnection therewith, whereby the display monitor is electrically connected with the camera and at least one LED provided in the distal end of the stylet tube;
(b) using the endotracheal tube exchanger to insert the stylet tube into a first endotracheal tube disposed in the trachea of a patient;
(c) disconnecting the stylet tube from the hand grip;
(d) removing the first endotracheal tube from the patient over the stylet tube;
(e) replacing the first endotracheal tube with a second endotracheal tube over the stylet tube;
(f) reconnecting the stylet tube to the hand grip;
(g) visualizing and adjusting as necessary the position of the second endotracheal tube using the display monitor on the hand grip and the at least one LED and camera provided adjacent the distal end of the stylet tube; and
(h) removing the stylet tube from the second endotracheal tube.

19. The method of claim 18, wherein, in step (a), the stylet tube includes a curved section proximate the distal end thereof.

20. The method of claim 18, wherein, in step (a), the end cap comprises a plurality of discrete contacts.

21. The method of claim 18, wherein, in step (a), the stylet assembly further comprises a PC board disposed in the stylet tube, the PC board positioned between, and in electrical contact with, each of the plurality of discrete contacts in the end cap and the camera and at least one LED proximate the distal end.

22. The method of claim 18, wherein, in step (a), the end cap of the stylet tube is matingly receivable in an electrical connection port defined in the support housing.

23. The method of claim 22, wherein, in step (a), the electrical connection port comprises a plurality of electrical contacts for electrical interconnection with the end cap.

24. The method of claim 18, wherein, in step (a), the end cap is removably mechanically connectable to the support housing.

25. The method of claim 23, wherein, in step (a), the display monitor is electrically connected through the support housing to the plurality of electrical contacts of the electrical connection port.

26. The method of claim 23, wherein, in step (a), the display monitor houses a power source for powering the display monitor and the camera and at least one LED disposed in the distal end of the stylet tube.

27. The method of claim 23, wherein, in step (a), the display monitor is removably mounted on an electrical connector provided on the support housing, the electrical connector being electrically connected through the support housing to the plurality of electrical contacts of the electrical connection port

28. The method of claim 23, wherein, in step (a), the endotracheal tube exchanger further comprises a hand grip connected to the support housing.

29. The method of claim 28, wherein, in step (a), the support housing is removably connected to the hand grip.
